(19) United States
(12) Patent Application Publication
(10) Pub. No.: US 2008/0081953 A1
(43) Pub. Date: Apr. 3, 2008

(54) Objective-subjective translation apparatus and methods

(76) Inventor: Peter Davis Poulsen, Grants Pass, OR (US)

Correspondence Address: DAVID S ALAVI, 2852 WILLAMETTE ST, #402, EUGENE, OR, 97405-8200, US

(21) Appl. No.: 11/073,268

(22) Filed: Mar. 3, 2005

Related U.S. Application Data
(60) Provisional application No. 60/549,635, filed on Mar. 3, 2004.

(51) Int. Cl. A61B 5/00

(52) U.S. Cl. 600/300

(57) ABSTRACT

A method comprises: selecting, for at least two analytical variables, a set of corresponding values so that the selected set of values is a solution of a predetermined analytical relationship among the analytical variables; and presenting to a user one or more sensory stimuli having at least two perceived sensory characteristics, each analytical variable having an assigned perceived sensory characteristic corresponding thereto, each corresponding sensory characteristic being related by a corresponding translation protocol to the corresponding selected solution value of the analytical variable. Each corresponding translation protocol establishes a substantially one-to-one relationship between corresponding values assumed by the analytical variable and the corresponding perceived sensory characteristic. The presented sensory stimuli evoke within the user an overall perception.

### BENEFIT CLAIMS TO RELATED APPLICATIONS

This application claims benefit of prior-filed co-pending provisional App. No. 60/549,635 entitled “Objective-subjective translator” filed Mar. 3, 2004 in the name of Peter David Poulsen, said provisional application being hereby incorporated by reference as if fully set forth herein.

### BACKGROUND

The field of the present system relates to objective-subjective translation. In particular, apparatus and methods are described herein for relating an objective quantity (i.e. the value of an analytic variable) to a subjective “quantity” (i.e. a perceived sensory characteristic).

### SUMMARY

A method comprises: selecting, for at least two analytical variables, a set of corresponding values so that the selected set of values is a solution of a predetermined analytical relationship among the analytical variables; and presenting to a user one or more sensory stimuli having at least two perceived sensory characteristics, each analytical variable having an assigned perceived sensory characteristic corresponding thereto, each corresponding sensory characteristic being related by a corresponding translation protocol to the corresponding selected solution value of the analytical variable. Each corresponding translation protocol establishes a substantially one-to-one relationship between corresponding values assumed by the analytical variable and the corresponding perceived sensory characteristic. The presented sensory stimuli evoke within the user an overall perception.

Objects and advantages pertaining to objective-subjective translation may become apparent upon referring to the exemplary embodiments illustrated in the drawings and disclosed in the following written description and/or claims.

### BRIEF DESCRIPTION OF THE DRAWINGS

FIG. 1 is a schematic overview of methods for objective-subjective translation.

FIG. 2 is a table illustrating an exemplary translation protocol.

FIG. 3 is a schematic diagram of an analytical relationship among analytical variables and a sensory translation of the variables.

FIG. 4 is a flow diagram for a game based on methods disclosed herein.

FIG. 5 is a process diagram for a game based on methods disclosed herein.

The embodiments shown in the Figures are exemplary, and should not be construed as limiting the scope of the present disclosure and/or appended claims.

### DETAILED DESCRIPTION OF EMBODIMENTS

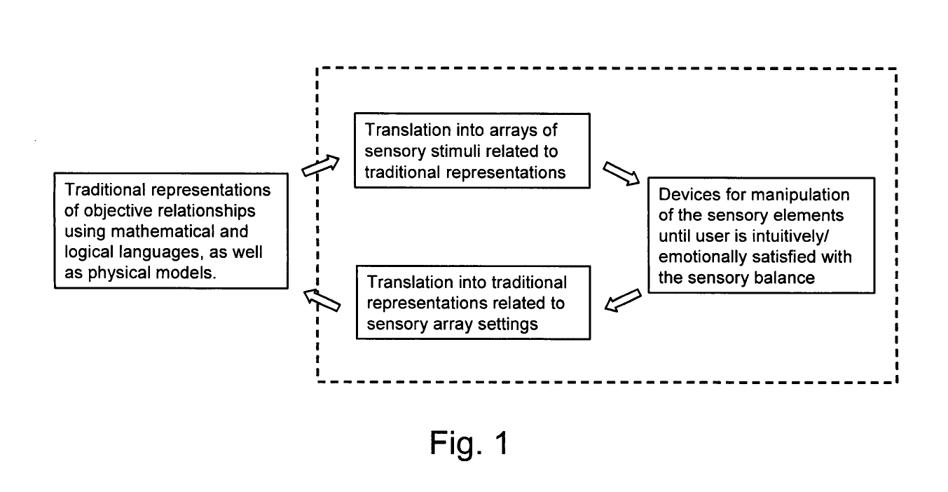

The system disclosed herein comprises a translation system. Quantitative or analytical relationships in physical systems or devices, between variables in mathematical equations, between elements of logic statements, or between other analytical variables (i.e. objective quantities) may be translated into, and out of, sensory-based variables (i.e. perceived sensory characteristics, or subjective “quantities”). Through use of the disclosed system, a user answers, by subjective means, objective questions associated with physical, mathematical, logical, or other analytical relationships. The system may include an interface for enabling subconscious rational processing elements of a neurological system to solve, via subjective means, problems whose natures have been thought previously to be addressable only along objective (or analytical or quantitative) avenues of solution. A schematic diagram of the basic system is shown in FIG. 1. Definitions of important terms used herein are listed below.

Analytical Problem: A physical, engineering, mathematical, logical, or other analytical, objective, or quantitative problem. Typically characterized by an analytical relationship, e.g. a system of equations or a set of logic statements. Once initialized, the analytical relationship may remain fixed as the system is used. Alternatively, it may be useful or desirable to allow the analytical relationship to change or evolve during use of the system.

Analytical Variables: The variables (numbers or words or symbols) within an analytical problem that may assume various values or states.

Analytical Solution: Solutions to an Analytical Problem, e.g. values for the analytical variables that form a solution of the analytical relationship.

Subjective (or Sensory) Problem: The Subjective or Sensory “translation” of an Analytical Problem. Typically would require a user to supply one or more missing Sensory Variables.

Sensory Variables: Perceived sensory characteristics of visual, audio, tactile, or other sensory stimuli that are typically each related to corresponding values of a corresponding analytical variable in a substantially one-to-one manner by a corresponding translation protocol. During the initialization of the system, each analytical variable is assigned to a corresponding perceived sensory characteristic, and a corresponding translation protocol is established. The assignment and translation protocol may remain fixed once initialized, or the assignment or translation protocol may change or evolve while the system is used, depending on the particular goals or objectives of use.

Subjective Solutions: A set of perceived sensory characteristics which, when translated into corresponding values for the analytical variables according to the corresponding translation protocols, are an analytical solution. Example Subjective Solutions may be presented in Training Cycles.

Subjective Solution Submission: The answers a user submits during a Problem Solving Cycle. They may be translated into values for the Analytical Variables and evaluated for correctness. One or more criteria may be imposed for determining whether a submitted solution is acceptable, and such criteria may be fixed or may vary. Such criteria may also be employed for selecting approximate example Subjective Solutions for Training Cycles, instead of always using “exact” solutions.

Subjective-Response Consistency: The feeling, emotion, or overall perception evoked within a user by performing a set of Training Cycles. It is typically intended that such an overall perception should remain substantially consistent within a particular set of Training Cycles. It is this overall perception that a user seeks to evoke when seeking a Subjective Solution.

Training Cycles: A set of example Subjective Solutions to a particular Analytical Problem that are presented to a user so that the user can achieve Subjective-Response Consistency.

Problem Solving Cycles: A set of Subjective Solutions presented to a user having one or more incorrect or missing values for their Sensory Variables. The user supplies or corrects these Sensory Variables, so as to evoke within the user substantially similar Subjective-Response Consistency as was evoked during the Training Cycles.

The system can be used with or without a set of Training Cycles. In either case, the system is initialized before the user begins. Initialization consists of assigning the Analytical Variables of an Analytical Problem to the corresponding Sensory Variables of a Subjective Solution, and establishing corresponding translation protocols. The Sensory Variables could include perceived sensory characteristics of visual, audio, tactile, and other sensory stimuli. It may often be the case that once initialized, each assigned sensory variable and its corresponding translation protocol may remain fixed to the corresponding analytical variable for the duration of use of the system. In other instances, however, it may be useful or desirable to allow the sensory variable assignment or corresponding translation protocol to change or evolve during use of the system. Such flexible variable assignments or translation protocols may enable a wider range of intuitive phenomena to be explored, evaluated, or quantified.

An example set of Sensory Variables might include a visual image comprising various visual elements: colors, brightness levels, textures, resolutions, oscillations, and so forth. Each Sensory Variable in a Subjective Problem is uniquely associated with an Analytical Variable in an Analytical Problem through a corresponding translation protocol that establishes a substantially one-to-one relationship between values assumed by the analytical variable and the corresponding perceived sensory characteristics (i.e. the sensory variable).

It is a characteristic of the system that a user need not know the correspondence or the translation protocol relating each of the Analytical Variables or the corresponding Sensory Variables. It may typically be desirable that the correspondence and the translation protocol not be known by the user at the outset, nor be discovered or discerned by the user during use. Such knowledge could yield conscious interference with the full utilization of the system as an intuitive system. Consciously discernible knowledge of the correspondence or the translation protocol relating the analytical and sensory variables, such that they form a correct solution, may interfere with the full value of the use of the system.

When Training Cycles are employed, the system presents a user with a series of example subjective solutions. In other words, the user is presented with sensory stimuli having corresponding perceived sensory characteristics related by the corresponding translation protocols to the corresponding values of the analytical variables that yield an analytical solution. These example subjective solution may be exact, or may be approximate within suitably chosen criteria (i.e. “acceptable” solutions, which incidentally shall also encompass the exact solutions). During the training process, users will develop, to greater and lesser degrees, an intuitive capacity (meaning rational but subconscious mental function) to have a substantially consistent response to each of the subjective solutions presented (i.e. a substantially consistent overall perception will be evoked within the user). The consistent response in the user may be identified as a feeling, impression, emotion (contentment, anger, sadness, tiredness, etc.), hunch, an overall perception, or other subjective mental/physiological manifestation.

It should be noted that it is not necessary that every user acquire the same impressions or overall perceptions from a given set of Training cycles. What is necessary is that an individual user must experience consistent feelings or overall perceptions (i.e. a Subjective-Response Consistency) within himself or herself for a particular set of Training Cycles. That is, the system requires Subjective-Response Consistency only within the current user and only for the duration of its use after a given initialization.

When the user of the system feels he or she has acquired Subjective-Response Consistency during the Training Cycle, he or she may begin one or more Problem-Solving Cycles. In this process, the system gives the user an opportunity to freely control, adjust, or select one or more of the Sensory Variables of the same sensory stimuli as were used for the Training Cycles. The user manipulates the Sensory Variable(s) until he or she finds a combination that evokes a common impression or overall perception substantially consistent with that evoked during the Training Cycles.

When the user is satisfied that he or she has adjusted the sensory variables (i.e. perceived sensory characteristics of the sensory stimuli) so as to evoke a consistent response, the choice is confirmed by making a Subjective Solution Submission. The Sensory Variables in the user's Subjective Solution Submission are then translated to their Analytical counterparts according to the corresponding translation protocols, and are evaluated to determine whether the translated values for the Analytical Variables are a solution to the Analytical Problem (equivalently, whether the adjusted Sensory Variable(s) are part of a solution to the counterpart Subjective Problem). One or more criteria may be employed for determining whether the submitted solution is acceptable. Solutions within a given range of an exact solution may be deemed acceptable, and the size of the range may be selected to achieve desired training results. The criteria may remain substantially fixed, or may vary during use of the system. For example, solutions within some predetermined percentage deviation (i.e. “error bars”) may be accepted, with the acceptable percentage deviation remaining constant throughout a session of use. Alternatively, the acceptable percentage deviation may be relatively larger at the beginning of a session (when the user is inexperienced), and then may decrease as the user becomes more proficient. Evolving criteria may change in a predetermined fashion or at a predetermined rate, or the evolution of the criteria may be linked to the user's performance in using the system. Solutions (acceptable or unacceptable) may be evaluated by engineers, physical scientists, biological scientists, behavioral scientists, or other analysts, or may be used for scoring in games that utilize the system.

The system may be adapted according to which sensory capabilities are available to a user. It is not necessary that all normal sensory capabilities be available to all users. The system as disclosed herein encompasses embodiments wherein sensory stimuli and corresponding translation protocols may be limited to less than the full array of human sensory functions. Accordingly, the system may be used by a deaf user, a blind user, a user lacking the ability to smell, or users with other sensory deficits or limitations, either singularly or in combination. Additionally, literacy, logical prowess, mathematical skill, or other traditional academic achievement is not necessary for a subject to successful utilize the system. Nor is it necessary that the user of the system be human. Therefore, it is also noted here that the system anticipates applicability to animals.

Assessment of a user's prowess in use of the system may be readily quantified. Simple statistical analysis of several trial cases may be used to determine whether the user has skills, either innate or the result of training, for intuitive or subjective solution of problems that have historically only been solvable by objective means. Such assessment must, however, account for several possibilities, including conscious rational processes that have determined the correct Sensory Variables. It should be noted that, and used herein, “intuition” shall denote rational activity occurring at a subconscious level. Or, if during the Problem-Solving Cycles the user chooses or adjusts the Sensory Variables to be the same as those presented in one of the Training Cycles, then it cannot be determined whether the user has memorized or has intuited the solution, or has by chance set the Sensory Variable values. If the combination of Sensory Variable values selected by the user is not among the training examples, then the subject has either intuited a solution, or has come upon the combination by chance. It is the intuited result that is engaged uniquely by the system disclosed herein.

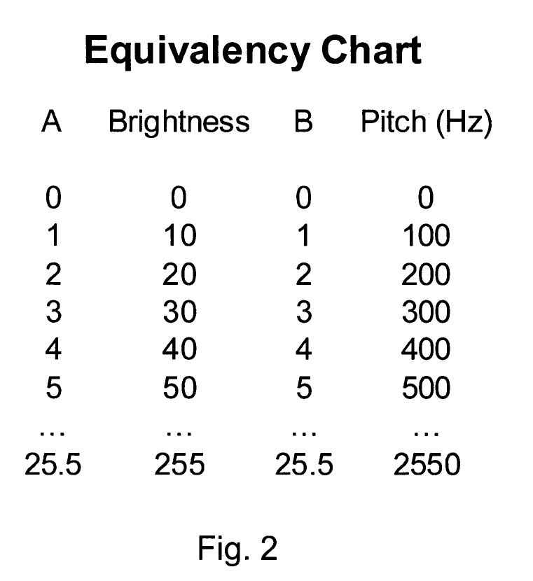

In a first exemplary embodiment, the analytical relationship A=B could be employed, with “A” and “B” as the Analytical Variables. When the system is initialized, “A” is assigned the Sensory Variable of screen brightness, and “B” the Sensory Variable of sound pitch. This could easily be done using a computer with a uniform screen having a brightness value that may be set between 0 and 255 (screen brightness units at 8-bit resolution), and having a sound system that may be made to generate sounds having pitch between 100 Hertz and 2550 Hertz. FIG. 2 shows simple translation protocols for converting between numerical values for A and B (the analytic variables) and perceived sensory characteristics of the sensory stimuli (brightness and pitch; the sensory variables).

In this example, after initialization, a user will begin a series of Training Cycles. The user will be presented with combinations of image brightness and sound pitch that exactly or approximately represent the conditions expressed by the equation. In other words, in each training cycle a combination of brightness and pitch is presented that corresponds to A=B from FIG. 2. For example, if the value of the analytical variable B is doubled, then the pitch of sound should double and the brightness of the image should double. As an exemplary set of training cycles, a user might be presented with the brightness/pitch combinations 100/1000, 50/500, 10/100 and 185/1850. The Training Cycles may continue for a predetermined number of repetitions after which the user would be assumed to be trained (i.e. expected to have achieved a Subjective-Response Consistency), or the training cycles may continue until the user declares himself or herself to be trained.

After the Training Cycles, a user may begin one or more Problem-Solving Cycles. A user is presented with one or the other of the Sensory Variables (in this example either the brightness corresponding to “A” or the pitch corresponding to “B”), and then chooses or adjusts the other Sensory Variable to achieve the same feeling or overall perception evoked during the Training Cycle. Once the user confirms his choice or adjustments (confirms his Subjective Solution Submission), the system translates the brightness and pitch back into their Analytical Variables and checks to see if they are an acceptable solution to the Analytical Problem. This checking may be made subject to any desired criterion, and a solution is typically deemed acceptable is it is within some selected error range around an exact solution. Such a criterion may be made as rigorous (e.g. only an exact match is acceptable) or as lax (e.g. any solution within an order or magnitude is acceptable) as dictated by the needs and objectives for a given use of the system. Such criteria may remain fixed during a given use of the system, or may vary or evolve as the system is used.

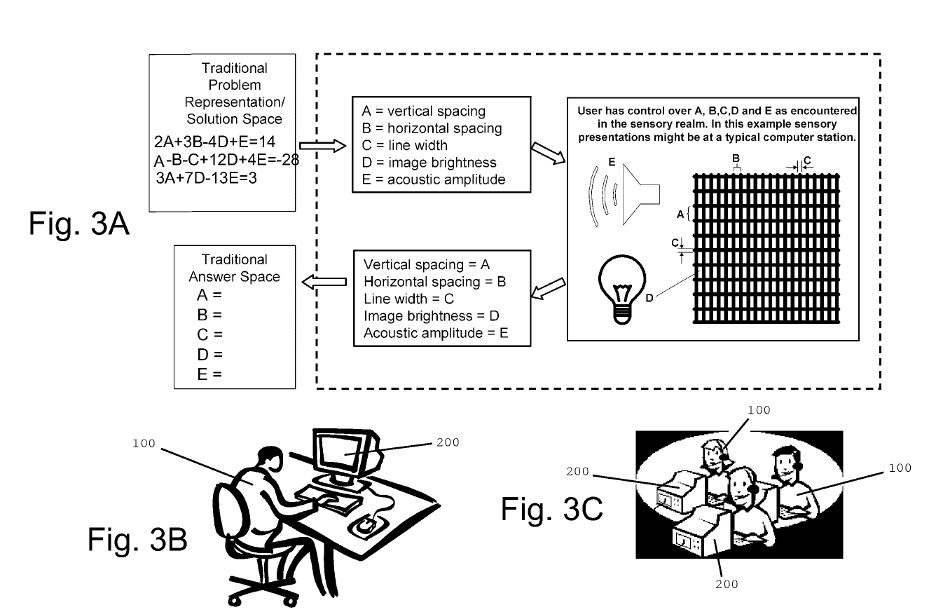

A second exemplary embodiment illustrated schematically in FIG. 3 is initialized to include an exemplary system of three linear equations as the analytical relationship among five analytic variables A, B, C, D, and E. The system translates these Analytical Variables into distinct Sensory Variables (i.e. perceived sensory characteristics of the sensory stimuli), in this example vertical grid spacing (A), horizontal grid spacing (B), gridline width (C), image brightness (D), and acoustic amplitude (E). The presentation of these Sensory Variables might, for example, be achieved at a typical computer work station, where the user would have control over one or more of the Sensory Variables as appropriate for the particular use of the system. Some other display device and/or other sound devices may be alternatively employed. During Training Cycles, a user would be presented with various sets of Sensory Variables (combinations of grid spacing, line width, brightness, and sound volume) that correspond, according to the corresponding translation protocols, to acceptable analytical solutions of the system of equations. During subsequent Testing Cycles, the user may be presented with four of the Sensory Variables already set, and the user may be asked to adjust the fifth Sensory Variable until it “feels” in the right balance with the four given elements (i.e. until an overall perception is evoked that is substantially consistent with that evoked during the Training Cycles, or equivalently until the user attains Subjective-Response Consistency). Once the user signals the system that a satisfactory Sensory Solution has been obtained (the user confirms his or her Subjective Solution Submission), the corresponding translation protocols are employed to find the associated analytical values, and the user is informed whether or not the values are an acceptable Analytical Solution. The user need not know anything about formal logic or mathematics to reach his subjective solution to the analytical problem.

Any set of equations or other analytical relationships may be employed. Depending on the particular analytical relationships chosen, it may sometimes be the case that one or more of the analytical variables (and hence the corresponding sensory variable) may vary freely, and one goal of the user's training is to come to recognize that one or more of the sensory variables may be unrelated to the problem. An example of such an analytical relationship would be a relationship between five variables A, B, C, D, and E defined by the three equations A*B=C, A/B=D, and C*D=E. It is easily seen that these equations reduce to A²=E, regardless of the values of B, C, or D. One indication of successful training would be a user beginning to ignore the sensory variables associated with the unrestricted analytical variables. This is only one example of the many and varied nuanced uses of the system and methods disclosed herein.

Another exemplary embodiment comprises a video-game implementation of the system. Such an embodiment might take commercial advantage of the social assessment that females generally are more intuitive than males (a prevalent perception, if not an actuality). A game using visual, audio, tactile, or other sensory elements that are interrelated by mathematical and/or logical equations may have far more appeal to female players than do the eye-hand-reflex, high-adrenalin, action games that appeal in greater proportion to males than to females. A video game based on more contemplative, creativity-based competition is made possible by the system.

Scoring with the system might be based on elements such as number or accuracy of Training Cycles needed, training time needed, time to reach a Subjective Solution, complexity of the Problem (Analytical and/or Subjective), or accuracy of the Subjective Solutions compared with the Analytical Solutions (i.e. solution acceptability criteria). An adventure game could, for example, be presented as a journey toward illumination or joy, with ever more difficult trials (problems) at various points along the way, perhaps also including various crossroads or decision points with specialized problem sets. Acceptability criteria for submitted solutions may become more rigorous as the game progresses, for example, or at one or more points there may be alteration of one or more of the sensory variables assigned to analytical variables, or alteration of one or more of the translation protocols relating the analytical and sensory variables. A graphical representation of the player progressing along the path to various milestones and towards some ultimate goal could be provided and would reflect the achievement relative to the successful application of the system at each trial.

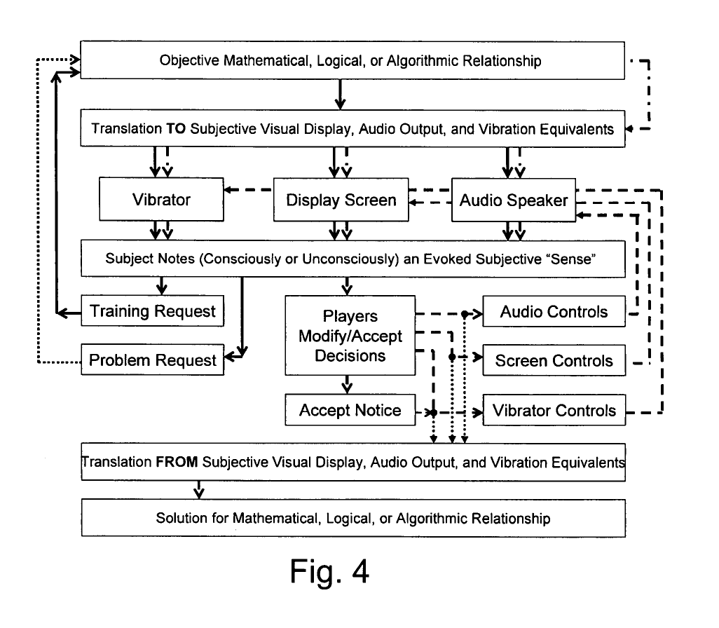

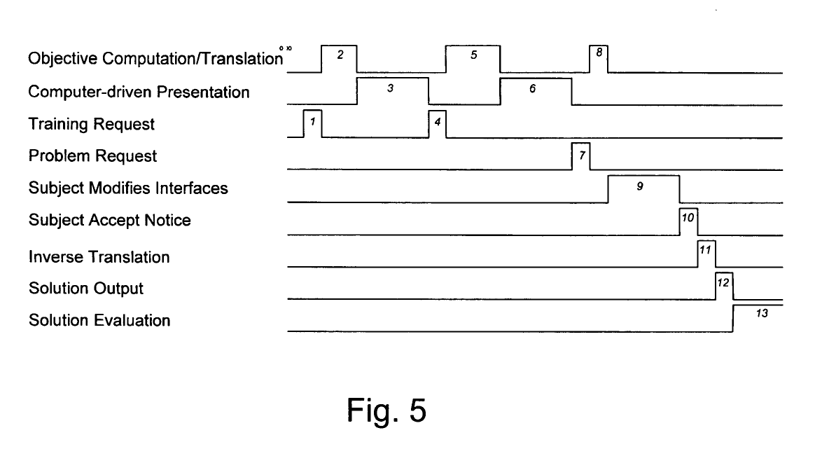

The flow diagram of FIG. 4 and the numbered sequencing diagram of FIG. 5 schematically illustrate operations within the video game and give the flow of events that happen “behind the scenes” of the game, but the Figures do not necessarily dictate the specifics of the game itself. In the flow diagram of FIG. 4 all flows associated with the Training process are represented by solid lines, while flows associated with the Problem Solving functions are represented by various forms of broken lines. Also, as stated earlier, the number of Sensory Variables possible for a game is far more than the three used in this illustration.

A player first signals for a Training Cycle, shown in the box labeled “Training Request” in FIG. 4, and the functional operation 1 going from “off” to “on” in FIG. 5. The training request is forwarded to the location within the system that maintains the problems, as represented by the box labeled “Objective Mathematical, Logical, or Algorithmic Relationship.” A Training Set of values for the analytical variables is selected (operation 2 of FIG. 5), and the selected values are mapped to their associated Sensory Variables according to the corresponding translation protocols (operation 2 of FIG. 5; the box labeled “Translation TO Subjective Visual Display, Audio Output, and Vibration Equivalents” in FIG. 4). Associated control signals are sent to various means for presenting the sensory stimuli with the corresponding perceived sensory characteristics (operation 3 of FIG. 5; the boxes labeled “Vibrator,” “Display Screen,” and “Audio Speaker” of FIG. 4). During the Training Cycles, the player attempts to achieve a Subjective-Response Consistency (box labeled “Subject Notes (Consciously or Unconsciously) an Evoked Subjective ‘Sense”’). As deemed appropriate by the player or by the system, additional Training Cycles may be presented (operations 4, 5, and 6 of FIG. 5; which may be repeated as needed, desired, or required).

Depending on the manner in which the system is initialized, Training Cycles continue until the player indicates to the system that he is ready to undertake a Problem-Solving Cycle, until a predetermined number of Training Cycles are presented, or until the system deems the player ready (by a suitably chosen criterion). A Problem Request (operation 7 of FIG. 5; the box labeled “Problem Request” in FIG. 4) initiates presentation of a Sensory Problem to be solved by the player. A set of values (perhaps incomplete or inconsistent) for the analytical variables is selected and translated according to the corresponding translation protocols to Sensory variables (operation 8 of FIG. 5; boxes labeled “Objective Mathematical, Logical, or Algorithmic Relationship” and “Translation TO Subjective Visual Display, Audio output, and Vibration Equivalents” in FIG. 4). The resulting translation displays proper values for only a subset of the Sensory Variables (boxes labeled “Vibrator”, “Display Screen”, and “Audio Speaker” in FIG. 4). The Sensory Variables not included in the properly-presented subset are those the player is tasked to supply. The player may or may not be told which Sensory Variables require choosing or adjustment. Or the subset of properly-presented Sensory Variables could be an empty set, requiring the player to determine (by choice or adjustment) all of the Sensory Variables required to form a Subjective Solution.

The player chooses or adjusts the Sensory Variables (operation 9 of FIG. 4; “control” boxes of FIG. 4) until he or she reaches the same Subjective-Response Consistency as he did during the Training Cycles (i.e., until an overall perception is evoked within the player that is substantially consistent with the overall perception evoked during the Training Cycles). When the player is satisfied that he or she has achieved the same Subjective-Response Consistency as during the Training Cycles, the player signals the system and makes a Subjective Solution Submission (operation 10 of FIG. 5; box labeled “Accept Notice” of FIG. 4). The system then translates the Subjective Values in the Subjective Solution Submission into their corresponding Analytical Values (operation 11 of FIG. 5; box labeled “Translation FROM Subjective Visual Display, Audio Output, and Vibration Equivalents” of FIG. 4) to yield a submitted candidate analytical solution (operation 12 of FIG. 5; box labeled “Solution for Mathematical, Logical, or Algorithmic Relationship” of FIG. 4). The Analytical Solution comprising values for the analytical variables translated from the submitted sensory variables are evaluated for accuracy (i.e., it is determined whether they are a solution to the analytical relationship). Based on the accuracy of the Solution, training time, number of Training Cycles, difficulty of the problem, or other criteria, the player's score is adjusted and the player's position along the graphically displayed journey may be advanced accordingly.

Multi-user implementations of the system and methods disclosed herein may be realized. This may be particularly desirable in a competitive gaming scenario. A system could be set up with each player acting simultaneously but substantially independently. Scores (based on any suitable criteria) may be compared during or at the end of a session. In more interactive implementations, the system could be configured so that each players actions may influence the conditions experienced by another player (variable assignments, translation protocols, acceptability criteria, and so forth). Such scenarios are manifold, and an exhaustive listing will not be attempted here. Such interactive implementations shall nevertheless fall within the scope of the present disclosure or appended claims.

In addition to gaming, other exemplary embodiments may be applied to areas including, but are not limited to, computing and engineering design efforts, card games, research tools for human cognition studies, psychiatric assessment, or other areas wherein intuitive problem-solving may be applicable.

As an example of a computing and engineering application, an objective engineering problem (a wing's lift-to-drag ratio, for example) could be submitted for solution to an individual who was not trained in the objective, traditional mathematical representation of the problem, but instead trained with training cycles that include known solutions to the engineering problem translated into corresponding sensory variables. Resultant solutions to the Analytical Problem, arrived at subjectively according to system and methods disclosed herein, may be correct and, by virtue of approaching the problem in a different “domain”, may provide unique and valuable solutions that may have been missed had only traditional analytical techniques been employed. For example, traditional analyses may suggest that the relationships in question only hold within some limited set of values for the analytical variables.

Card game embodiments of the system may be implemented. For example, a series of individual pictures could be printed on cards. Each of the pictures would be a graphic representation of individual solutions to a specific analytical equation. That is, the system would translate the analytical variables into pictorial lines, line widths, textures, spacing, color, brightness, orientation, resolution, etc. These cards, each one an accurately balanced sensory presentation of the objective relationship associated with the equation (or set of equations) would be used during a training period where the player is free to examine as many training cards as desired. A card player's training period could be measured in terms of elapsed time, in terms of number of cards viewed, or both, as well as in some other terms. Once the player was satisfied that a common subjective impression had been reached for the training cards, then the player would be presented with a stack of test cards. The test cards would incorporate various graphic relationships. Many of the included pictures would be related to the equation governing the training cards, but with varying degrees of accuracy. The player would be challenged to sort through the test cards to choose the most accurate of the cards, perhaps setting aside the top two or three selections according to the perceived accuracy of each. Then the cards would be checked for accuracy against the equation via some form of reference. Players would be scored according to the collective accuracies and order of their selections, as well as by the amount of time needed to train. A card game version might incorporate some form of player exchange of cards. Alternatively, a large graphic of training images could be set in the center of play and the players could draw and exchange cards seeking that card that seems to meet the balance. Many variations of card games utilizing the system and methods disclosed herein could be implemented, and all shall fall within the scope of the present disclosure or appended claims.

In another example, the system and methods disclosed herein could be used as a research tool for human cognition studies. The system and methods disclosed herein could also be used as a psychiatric assessment tool by comparing the subjective balances selected by a patient with a set of normative balances for the population in general, or sets of norms for various subsets of the population. Some of the subsets would include groups with specific gifts while other subsets would include groups with specific emotional problems. Many other fields could benefit from problem-solving and assessment capabilities of the system and methods disclosed herein. It is intended that all such uses shall fall within the scope of the present disclosure or appended claims.

It should be noted that the system and methods disclosed herein may be applied to any number of analytical variables and corresponding sensory variables. It should be noted that the sensory variables (i.e. the perceived sensory characteristics of the sensory stimuli) may apply to any one or more of the five senses. In a given embodiment, all the sensory variables may apply to only one of the senses, or each might apply to a different sense, or some senses may have multiple sensory variables while other senses have only one or have none. Any suitable translation protocol may be employed for relating a value of an analytical variable to a corresponding perceived sensory characteristic of a sensory stimulus. It may typically be the case, but is not necessarily always the case, that a sensory characteristic may be numerically quantifiable (e.g. a brightness level, a loudness level, a line spacing, vibration frequency, and so forth), so that the sensory characteristic may be readily translated to a numeric value for an analytical variable and vice versa. However, the system and methods disclosed herein are not limited to such quantifiable/numerical examples, and shall encompass any suitable analytical variable types, suitable sensory characteristics, and suitable translation protocols yielding substantially one-to-one relationships between analytical variables and corresponding sensory characteristics. It should be noted that such substantially one-to-one relationships would obtain at any given time. However, alteration of an assigned sensory variable or alteration of a translation protocol, while yielding a new substantially one-to-one relationship after the alteration, would not necessarily preserve a substantially one-to-one relationship that encompasses the analytical and corresponding sensory variables both before and after the alteration. Such embodiments shall nevertheless fall within the scope of the present disclosure or appended claims.

Any suitable sensory characteristics may be employed as a Sensory Variable. Suitable sensory characteristics may be perceived via any of the senses (sight, hearing, touch, taste, smell). The following examples of perceived sensory characteristics for sensory stimuli comprise only a small sampling of sensory characteristics that might be employed within the scope of the present disclosure or appended claims; the scope of the present disclosure or appended claims shall in no way be limited to those examples recited herein. Examples of perceived sensory characteristics of an auditory stimulus may include pitch, volume, timbre, harmonics, overtones, dissonance, resonance, modulation amplitude, modulation frequency, modulation pattern, or beat frequency. Examples of perceived sensory characteristics of a visual stimulus may include brightness, color, hue, tint, color gradient, color mixture, modulation amplitude, modulation frequency, modulation pattern, or granularity. Examples of perceived sensory characteristics of a geometric pattern (a visual stimulus) may include grid size, grid orientation, grid angle, periodicity, line thickness, line density, line color, line type, line brightness, fill color, fill pattern, fill brightness, translational invariance, rotational symmetry, or reflection symmetry. Examples of perceived sensory characteristics of a tactile stimulus may include texture, roughness, temperature, vibration amplitude, vibration frequency, or vibration modulation.

It should be noted that while computer-based displays and audio systems have been disclosed in various exemplary embodiments as means for presenting sensory variables to a user, the present disclosure is not limited to such computer-based means. Any visual display system, audio system, mechanical system, or other system may be employed for presenting sensory stimuli to a user, and for enabling a user to adjust one or more perceived sensory characteristics of the sensory stimuli.

For purposes of the present disclosure and appended claims, the conjunction “or” is to be construed inclusively (e.g., “a dog or a cat” would be interpreted as “a dog, or a cat, or both”; e.g., “a dog, a cat, or a mouse” would be interpreted as “a dog, or a cat, or a mouse, or any two, or all three”), unless: i) it is explicitly stated otherwise, e.g., by use of “either. . . or”, “only one of . . . ”, or similar language; or ii) two or more of the listed alternatives are mutually exclusive within the particular context, in which case “or” would encompass only those combinations involving non-mutually-exclusive alternatives. It is intended that equivalents of the disclosed exemplary embodiments and methods shall fall within the scope of the present disclosure and/or appended claims. It is intended that the disclosed exemplary embodiments and methods, and equivalents thereof, may be modified while remaining within the scope of the present disclosure or appended claims.

1. A method, comprising:
selecting, for at least two analytical variables, a first set of corresponding values so that the first selected set of values is a first acceptable solution of a predetermined analytical relationship among the analytical variables;
presenting to a user a first set of one or more sensory stimuli, the set having at least two perceived sensory characteristics, each analytical variable having an assigned perceived sensory characteristic corresponding thereto, each corresponding perceived sensory characteristic being related by a corresponding translation protocol to the corresponding first selected solution value of the corresponding analytical variable;
selecting a second set of corresponding values for the analytical variables so that the second set of values is a second acceptable solution of the analytical relationship between the analytical variables; and
presenting to the user a second set of the sensory stimuli having the corresponding perceived sensory characteristics related by the corresponding translation protocols to the corresponding second selected set of solution values of the analytical variables
wherein:
each corresponding translation protocol establishes a one-to-one relationship between corresponding values assumed by the corresponding analytical variable and the corresponding perceived sensory characteristic; and
the second set of sensory stimuli evokes within the user an overall perception recognizable by the user as consistent with an overall perception evoked within the user by the first set of sensory stimuli.

2. (canceled)

3. The method of claim 1, wherein the second set of sensory stimuli having corresponding perceived sensory characteristics related to the corresponding second set of selected solution values are presented in response to a request from the user.

4. The method of claim 1, further comprising altering the analytical relationship prior to selecting the second set of corresponding values for the analytical variables, so that the second set of values is an acceptable solution of the altered analytical relationship between the analytical variables

5. The method of claim 1, further comprising altering at least one translation protocol prior to presenting to the user the second set of sensory stimuli having corresponding perceived sensory characteristics related by the corresponding translation protocols to the corresponding second set of selected solution values of the analytical variables.

6. The method of claim 1, further comprising:
enabling the user to choose or adjust at least one corresponding perceived sensory characteristic of the sensory stimuli;
presenting to the user a new set of the sensory stimuli having the corresponding perceived sensory characteristics related by the corresponding translation protocols to a corresponding proposed set of solution values for the analytical variables,
instructing the user to choose or adjust at least one corresponding perceived sensory characteristic of the new set of sensory stimuli so that the user perceives that the new set of sensory stimuli evokes within the user an overall perception consistent with the overall perception evoked within the user by the first and second sets of sensory stimuli;
determining, according to the translation protocol, a set of corresponding values for the analytical variables based on the corresponding chosen or adjusted perceived sensory characteristics of the sensory stimuli, and determining whether the set of values thus determined is an acceptable solution of the analytical relationship; and
informing the user as to whether the determined set of values is an acceptable solution of the analytical relationship.

7. The method of claim 6, further comprising altering a criterion for determining whether the determined set of values is an acceptable solution of the analytical relationship.

8. The method of claim 1, wherein at least one of the sensory stimuli is an auditory stimulus.

9. The method of claim 8, wherein at least one perceived sensory characteristic of the auditory stimulus is pitch, volume, timbre, harmonics, overtones, dissonance, resonance, modulation amplitude, modulation frequency, modulation pattern, or beat frequency.

10. The method of claim 1, wherein at least one of the sensory stimuli is a visual stimulus.

11. The method of claim 10, wherein at least one perceived sensory characteristic of the visual stimulus is brightness, color, hue, tint, color gradient, color mixture, modulation amplitude, modulation frequency, modulation pattern, or granularity.

12. The method of claim 10, wherein the visual stimulus comprises a geometric pattern, and the perceived sensory characteristic of the geometric pattern is grid spacing, grid orientation, grid angle, periodicity, line thickness, line density, line color, line type, line brightness, fill color, fill pattern, fill brightness, translational invariance, rotational symmetry, or reflection symmetry.

13. The method of claim 1, wherein at least one of the sensory stimuli is a tactile stimulus.

14. The method of claim 13, wherein at least one perceived sensory characteristic of the tactile stimulus is texture, roughness, temperature, vibration amplitude, vibration frequency, or vibration modulation.

15. The method of claim 1, wherein sensory stimuli are presented to multiple users simultaneously.

16-30. (canceled)
